(19) United States
(12) Patent Application Publication
(10) Pub. No.: US 2021/0186836 A1
(43) Pub. Date: Jun. 24, 2021

(54) COSMETIC COMPOSITIONS COMPRISING SPECIAL CARBODIIMIDES, FOR HAIR

(71) Applicant: Covestro Deutschland AG, Leverkusen (DE)

(72) Inventors: Sebastian Doerr, Düsseldorf (DE); Sophia Viala, Köln (DE); Laurence Pottie, Köln (DE); Paula Cristina Alves Rodrigues, Düsseldorf (DE)

(21) Appl. No.: 16/499,521

(22) PCT Filed: Mar. 29, 2018
(86) PCT No.: PCT/EP2018/058133

§ 371 Date: Sep. 30, 2019

(30) Foreign Application Priority Data
Apr. 3, 2017 (EP) 17164589.8

(51) Int. Cl. A61K 8/40; A61K 8/87; A61Q 5/06; A45D 19/00

(52) U.S. Cl. CPC A61K 8/40; A61K 8/87; A61Q 5/06; A45D 19/0075; A61K 2800/95; A61K 2800/5428; A61K 2800/5426; A61K 2800/49; A61K 2800/5424

(57) ABSTRACT

The invention relates to a cosmetic composition for hair, containing at least one compound containing at least three carbodiimide groups built according to formula (I), R⁴—[R¹—N═C═N]ₙ—R⁵ (I), where n is a whole number between 3 and 100, and R¹, R⁴ and R⁵ each stand, independently of one another, for linear or branched, aliphatic or cycloaliphatic, optionally substituted groups with 1 to 100 C-atoms. The invention also relates to uses of the cosmetic composition for hair, and to a method and the application thereof.

The present invention relates to hair cosmetics compositions, also referred to hereinafter merely as cosmetic compositions, containing carbodiimides, especially polycarbodiimides or aqueous dispersions thereof, for hair design, to uses thereof and methods for application thereof.

Products known as hair fixatives are used to design and stabilize a variety of hairstyles. Hair fixatives are usually in the form of mousses or hairsprays of barely differing composition. Mousses are applied to damp hair as an aid to model the hairstyle. By contrast, hairsprays are applied to dry ready-styled hair to fix the hairstyle.

In the case of hairsprays and mousses, the means of fixing or designing the hairstyle are usually in the form of aerosol containers, squeeze bottles or preparations sprayable by a pumping, spraying or foaming devices, consisting of an alcoholic or aqueous-alcoholic solution of film-forming natural or synthetic polymers. These polymers may be selected from the group of nonionic, cationic, amphoteric or anionic polymers.

A cosmetic composition such as that of the invention can serve to fix hair designs, but, optionally together with additives, can also serve for the decorative design, especially coloring, of the hair, especially of the eyelids and the eyebrows. The composition of the invention preferably makes the decorative effect longer-lasting, protects it against contamination and makes it more water-resistant. The cosmetic composition of the invention may, for example, at least be part of a hair dye, a hair fixative, a hair shampoo, a mascara or other hairstyling products. The aforementioned list of decorative products is of course nonlimiting.

Consumers naturally desire a long-lasting cosmetic effect when using cosmetic formulations. In particular, consumers expect good resistance to water, such as during bathing or showering, and to tears or sweat, especially during sports activities.

The stability of cosmetic products to water, tears or sweat (often called water resistance) is frequently improved using film-forming polymers. Film-forming polymers selected are preferably polymers based on acrylates or vinylpyrrolidones. The disadvantages of such film-forming polymers are known to the person skilled in the art. One is that the acrylate polymers form hard and brittle films. This results in an unpleasant feeling while wearing the product. Because of the sticky feel, the vinylpyrrolidones can only be used in limited concentrations.

The use of polyurethane dispersions is known in cosmetic and decorative cosmetics. EP 1010418 describes the use of an aqueous polyurethane dispersion in a wax-free mascara composition, but not in combination with carbodiimides. Monomeric carbodiimides have already been described for treatment of hair. Thus, DE 1 617 710 A describes the use of carbodiimides for improving the histological properties of hair after dyeing. For this purpose, mono- and dicarbodiimides that are water-soluble are used. There is no description here of a long-lasting effect of carbodiimides on hair.

There is thus still room for improvement in terms of wear comfort, especially reduced tackiness, stability, especially water stability, and improved wetting characteristics, for example to improve soiling characteristics, especially in combination with good compatibility with other cosmetic agents in the prior art cosmetic compositions.

It is therefore an object of the present invention to reduce at least one disadvantage of the prior art at least to a degree.

It is a further object of the invention to provide a cosmetics composition having high wear comfort, especially reduced stickiness, high stability, especially moisture stability and water stability, and improved wetting characteristics. In addition, the aqueous dispersions used in accordance with the invention should have comparatively low viscosity, so that they can easily be incorporated into cosmetic compositions for various purposes, for example for achieving decorative effects, but also for structure-conserving purposes.

It was an object of the invention to provide a cosmetics composition, especially a hair cosmetics composition, that has pleasant tactile properties on the hair, and especially does not feel tacky or lumpy, coupled with good fixing of the hair structure.

It was additionally an object of the invention to provide for various uses of the compositions of the invention for conserving the structure of shaped hair.

It was a further object of the invention to provide various uses of the composition of the invention for conserving the structure of shaped hair, wherein the treated hair retains its structure for as long as possible, especially in contact with high air humidity, water, shampoo or all of these.

It was a further object to provide a cosmetic method that enables achievement of a structure-conserving effect on shaped hair, wherein the treated hair retains its structure for as long as possible, especially in contact with water, shampoo or both.

At least one of the objects is surprisingly achieved by a hair cosmetics composition comprising at least one compound containing at least three carbodiimide groups and having a structure conforming to the formula (I)

R⁴—[R¹—N═C═N]ₙ—R⁵ (I)

where

n is an integer from 3 to 100, preferably from 3 to 50, or preferably from 3 to 20, or preferably from 3 to 10,

R¹ and R⁵ are each independently linear or branched, aliphatic or cycloaliphatic, optionally substituted groups having 4 to 100, or preferably from 4 to 50, or preferably from 4 to 20, carbon atoms.

R⁴ represents linear or branched, aliphatic or cycloaliphatic, optionally substituted groups having 1 to 100, or preferably from 1 to 50, or preferably from 1 to 20, carbon atoms.

Useful substituents of the R⁴ and/or R⁵ radicals in the context of the invention include numerous organic groups, for example alkyl, cycloalkyl, aryl, alkoxy, halogen, ether, thioether, thiourethane, disulfide, sulfoxide, sulfone, sulfonate, amino, aldehyde, keto, carboxylic ester, carboxylic acid, carbonate, carboxylate, cyano, alkylsilane and alkoxysilane groups, urethane groups, allophanate groups, biuret groups, urea groups and carboxylamide groups. In particular, a portion, also called a substituent, of the R¹, R⁴ or R⁵ radicals, preferably of the R⁴ and/or R⁵ radicals, is selected from the group consisting of a polyethylene glycol group, a polypropylene group, a methoxy group, an ethoxy group, a butoxy group, a methoxy polyethylene glycol group, a urethane group, an alkyl group, a cycloalkyl group, or a combination of at least two of these.

Further preferably, at least one of the R¹, R⁴ or R⁵ radicals, preferably the R⁴ and/or R⁵ radicals, has both an alkyl group and a cycloalkyl group. In addition, it is preferable that at least one of, more preferably both of, the R⁴ and R⁵ radicals has at least one of the groups, preferably at least two of the groups, selected from the group consisting of a urethane group, a polyethylene glycol group (PEG), a polypropylene group (PPG), a methoxy group, an ethoxy group, an alkyl group and a cycloalkyl group or a combination of at least two of these. In particular, it is preferable that the R⁴ and/or R⁵ radicals have an alkyl group, a cycloalkyl group, a urethane group, a PEG group and a methoxy group, preferably in exactly that order. Both R⁴ and/or R⁵ radicals preferably contain at least one urethane group and at least one group which is a radical derived from a C₁ to C₃₀ alcohol or a C₁ to C₃₀ monoalkoxy ethylene glycol.

A hair cosmetics composition according to the invention is understood to mean a composition suitable for achieving a desired effect on hair treated with the hair cosmetics composition, but not directly suitable in the same way for a use in skin cosmetics. In addition, a hair cosmetics composition is understood to mean a composition that has only slight adverse effects on the hair, and preferably none, and when employed causes minimum irritation or any other adverse effect to the areas of the skin that come into contact with the composition. In the context of the invention, an adverse effect is understood to mean in particular that the skin dries out, becomes cracked or reddened, develops rashes, urticaria or any other allergic reaction, or a layer in formed on the skin that cannot be washed off.

The compound containing at least three carbodiimide groups, also referred to hereinafter as polycarbodiimide, preferably comprises water-soluble or water-dispersible compounds.

The polycarbodiimide present in the composition of the present invention preferably has a molecular weight M_{w} (determined by GPC) within a range from 300 to 500 000 g/mol, or preferably within a range from 500 to 300 000 g/mol, or preferably within a range from 1000 to 200 000 g/mol.

A preferred process for preparation of aqueous dispersions of polycarbodiimides comprises at least one step, wherein, in the at least one step, at least one aliphatic or cycloaliphatic polyisocyanate is converted at a temperature in the range from 160 to 230° C. in the presence of carbodiimidization catalyst to a polycarbodiimide having an average functionality greater than 3, preferably from 3 to 100, or preferably from 3 to 50, or preferably from 3 to 20, or preferably from 3 to 10, carbodiimide units. Preferably, the reaction gases are temporarily or permanently removed. The amount of carbodiimidization catalyst is preferably 50 to 3000 ppm, based on the molar amount of polyisocyanate.

For the purposes of the present invention, the average functionality of carbodiimide units means the average number of carbodiimide units. The average functionality can also be a fraction. The average functionality is preferably 3 to 50, or preferably 3 to 20, or preferably 3 to 10. The higher the functionality, the lower the dispersibility of the hydrophilized polycarbodiimide in water.

In the preparation of the polycarbodiimide, preference is given to selecting an aliphatic or cycloaliphatic polyisocyanate from the group consisting of methylene diisocyanate, dimethylene diisocyanate, trimethylene diisocyanate, tetramethylene diisocyanate, pentamethylene diisocyanate, dipropyl ether diisocyanate, 2,2-dimethylpentane diisocyanate, 3-methoxyhexane diisocyanate, octamethylene diisocyanate, 2,2,4-trimethylpentane diisocyanate, nonamethylene diisocyanate, decamethylene diisocyanate, 3-butoxyhexane diisocyanate, 1,4-butylene glycol dipropyl ether diisocyanate, thiodihexyl diisocyanate, metaxylylene diisocyanate, paraxylylene diisocyanate, tetramethylxylylene diisocyanate, dicyclohexylmethane 4,4′-diisocyanate (H12MDI), isophorone diisocyanate (IPDI), hexamethylene diisocyanate (HDI), hydrogenated xylylene diisocyanate (H6XDI), 1,12-diisocyanate dodecane (DDI), norbornane diisocyanate (NBDI) and 2,4-bis(8-isocyanatoctyl)-1,3-dioctylcyclobutane (OCDI) or a mixture of at least two of these. Particular preference is given to isophorone diisocyanate (IPDI), hexamethylene diisocyanate (HDI), hydrogenated xylylene diisocyanate (H6XDI), dicyclohexylmethane 4,4′-diisocyanate (H12MDI). Most preferably, the cycloaliphatic polyisocyanate is dicyclohexylmethane 4,4′-diisocyanate (H12MDI).

The carbodiimidization catalyst is preferably an organophosphorus compound, more preferably organophosphorus compounds selected from the group consisting of phosphine oxide, phospholane oxide and phospholene oxide, and their sulfo and imino analogs. The phosphine, phospholene and phospholane oxides, sulfides and imino derivatives can be generated in situ, inter alia, from corresponding precursors with trivalent phosphorus, such as phosphines, phospholanes and phospholenes. The phospholene oxide is preferably selected from the group consisting of 3-methyl-1-phenyl-2-phospholene 1-oxide, 3-methyl-1-ethyl-2-phospholene 1-oxide, 1,3-dimethyl-2-phospholene 1-oxide, 1-phenyl-2-phospholene 1-oxide, 1-ethyl-2-phospholene 1-oxide, 1-methyl-2-phospholene 1-oxide. Further suitable catalysts and preferred embodiments of the process for the preparation of the polycarbodiimide are described in WO 2011/120928 A2.

A preferred process for preparing aqueous dispersions of polycarbodiimide comprises the steps of:
a) converting at least one aliphatic or cycloaliphatic polyisocyanate at a temperature in the range of 160 to 230° C. in the presence of 50 to 3000 ppm (ppm=parts per million, 1 ppm=0.0001%, molar proportions) of carbodiimidization catalyst, based on the molar amount of polyisocyanate, to a polycarbodiimide, preferably having an average functionality of 3 to 20 carbodiimide units, is reacted, wherein the reaction gases are temporarily or permanently specifically removed from the reaction medium,
b) reacting the polycarbodiimide obtained in step a) with at least one hydrophilic compound that bears at least group reactive toward isocyanate and/or carbodiimide groups, for example but not limited to those selected from the group consisting of polyethoxymonools, polyethoxydiols, polyethoxypolypropoxymonools, polyethoxypolypropoxydiols, polyethoxymonoamines, polyethoxydiamines, polyethoxypolypropoxymonoamines, polyethoxypolypropoxydiamines, hydroxyalkylsulfonates, aminoalkylsulfonates, polyethoxymono- and -dithiols, polyethoxymono- and -dicarboxylic acids, mono- and dihydroxycarboxylic acids or salts thereof to form a hydrophilized carbodiimide,

optionally further reacting the unreacted isocyanate groups with further compounds reactive toward isocyanate groups, for example with water, alcohols, thiols, amines, mineral acids and carboxylic acids, and
c) dispersing the compound obtained in step b) in water.

Preferably, the polycarbodiimide containing isocyanate groups which is obtained in step a) is converted according to the present invention in such a way that 10 to 70 mole percent (mol %) of the isocyanate groups present in the polycarbodiimide are reacted with at least one hydrophilic compound as component step b1) of step b), where the hydrophilic compound is selected from the group consisting of polyethoxymonools, polyethoxydiols, polyethoxypolypropoxymonools, polyethoxypolypropoxydiols, polyethoxymonoamines, polyethoxydiamines, polyethoxypolypropoxymonoamines, polyethoxypolypropoxydiamines, hydroxyalkylsulfonates, aminoalkylsulfonates, polyethoxymono- and -dithiols, polyethoxymono- and -dicarboxylic acids. In component step b2) of step b), 30 to 90 mol % of the remaining isocyanate groups are then reacted with at least one compound reactive toward isocyanate groups, for example polyethoxymonools, polyethoxydiols, polyethoxypolypropoxymonools, polyethoxypolypropoxydiols, polyethoxymonoamines, polyethoxydiamines, polyethoxypolypropoxymonoamines, polyethoxypolypropoxydiamines, hydroxyalkylsulfonates, aminoalkylsulfonates, polyethoxymono- and -dithiols, polyethoxymono- and -dicarboxylic acids, water, C₁ to C₃₀ alcohols, C₁ to C₃₀ thiols, amines, mineral acids and carboxylic acids.

The polycarbodiimides are preferably reacted in component step b1) of step b) with at least one compound selected from the group of compounds conforming to the general formula (III):

R⁶—O—(CH₂— CH₂—O)ₘ—H (III)

with R⁶=C₁ to C₃₀ alkyl or acyl group and m=4 to 30,

more preferably with at least one compound selected from the group conforming to the formula (III), where R 6 is a methyl group and m=10 to 30.

Very particular preference is given to monomethoxy polyethylene glycol with m=15-20.

Preferred C1 to C30 alcohols which can be used for further reaction of the isocyanate groups present in the polycarbodiimide that have not been fully reacted with the hydrophilic compounds in component step b2) of step b) are firstly water, low molecular weight monoalcohols, or else diols having a molecular weight of preferably from 32 to 500, more preferably from 62 to 300, g/mol. Very particular preference is given to using short-chain monoalcohols, i.e. branched or unbranched monoalcohols having 1 to 30 carbon atoms, such as methanol, ethanol, propanol, 1-butanol, 1-pentanol, 1-hexanol, cyclohexanol, cyclohexylmethanol, 2-ethylhexanol, dodecanol, stearyl alcohol or oleyl alcohol, mixtures thereof with one another and mixtures of isomers thereof and short-chain dialcohols having 2 to 60 carbon atoms, such as butane-1,4-diol, pentane-1,5-diol, hexane-1,6-diol, cyclohexanediol, cyclohexanedimethanol, octane-1,8-diol, nonane-1,9-diol, decane-1,10-diol, undecane-1,11-diol, dodecane-1,12-diol, tridecane-1,13-diol, tricyclodecanedimethanol, the mixture thereof with one another and mixtures of their isomers.

The sequence of component steps b1) and b2) of step b) may be fixed so that component step b1) may precede component step b2), they may be simultaneous or the order may be reversed.

Optionally, the aqueous dispersion of hydrophilized polycarbodiimide is adjusted in a step d) to a pH in the range of 7 to 12 (at 23° C.), more preferably in the range of 8 to 11. For this purpose, it is possible to use amine solutions, bases and conventional buffer solutions.

The median particle size d50 of the water-dispersed polycarbodiimide particles is typically in the range from 5 to 500 nm, preferably from 15 to 200 nm, more preferably from 25 to 100 nm. The median particle size d50 is the diameter above which and below which are 50% by weight of the particles. It can be determined by ultracentrifuge measurement (W. Scholtan, H. Lange, Kolloid, Z. und Z. Polymere 250 (1972), 782-796). The average particle sizes and particle size distributions can also be determined by light scattering methods which, although less precise, can correlate very well with the ultracentrifugation measurements, provided that there are no polymodal or very broad particle size distributions.

In a preferred embodiment of the hair cosmetics composition, the compound containing at least three carbodiimide groups has the general formula (II):

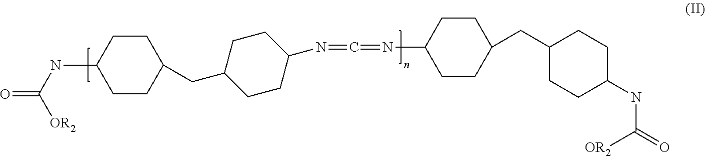

in which R₂ and R₃ are independently a radical derived from a compound selected from the group consisting of a monoalkoxy poly(ethylene glycol) of the general formula (III)

R⁶—O—(CH₂— CH₂—O)ₘ—H (III)

with R⁶=C₁ to C₃₀ alkyl or acyl group and m=4 to 30,

and a radical derived from C₁ to C₃₀ alcohol or a C₁ to C₃₀ monoalkoxy ethylene glycol.

The aqueous polycarbodiimide dispersions and/or solutions prepared by the process described above typically have a solids content of from 10% to 80% by weight, preferably from 20% to 60% by weight and more preferably from 30% to 50% by weight.

In a preferred embodiment of the hair cosmetics composition, the composition contains the compound containing the carbodiimide, also called polycarbodiimide, of the formula (I) or (III) within a range from 0.01% to 50% by weight, preferably within a range from 0.05% to 30% by weight, or preferably within a range from 0.1% to 20% by weight, or preferably within a range from 0.5% to 10% by weight, based on the total weight of the cosmetic composition. Depending on the use of the cosmetic composition, for example in hair application products, the preferred amounts of polycarbodiimides can vary widely. Hair application products are products such as hairstyling, haircare or hair coloring products. In addition, the cosmetic composition may also include coloring matter and a mascara, a shampoo, a setting agent, a colorant, each of which may be in the form of or be part of a spray, lotion, cream, foam, solution, emulsion or wax. The cosmetic composition preferably contains the polycarbodiimide within a range from 0.1% to 10% by weight, or preferably within a range from 0.2% to 8% by weight, or preferably within a range from 0.5% to 5% by weight, based on the total weight of the cosmetic composition, for example in shampoos. The cosmetic composition preferably contains the polycarbodiimide within a range from 0.5% to 30% by weight, or preferably within a range from 1% to 20% by weight, or preferably within a range from 2% to 15% by weight, based on the total weight of the cosmetic composition, in hair dyeing products.

In a preferred embodiment of the hair cosmetics composition, the composition further comprises at least one of the following components:
K1. at least one cosmetically active substance and/or one or more cosmetic auxiliary substances,
K2. at least one solvent or thinner,
K3. at least one film former.

The composition of the invention preferably comprises one or more cosmetic auxiliary substances that are customary in cosmetics, such as antioxidants, light stabilizers and/or other auxiliaries and additives, for example emulsifiers, wetting agents such as interface-active substances, plasticizers such as glycerol, glycol and phthalate esters and ethers, defoamers, thickeners and rheology modifiers, gelling agents, antisticking agents, surfactants, active ingredients, humectants, fillers, UV filters, film formers, solvents, coalescents, aromas, odor absorbers, fragrances and perfumes, gel formers and/or other polymer dispersions, for example dispersions based on polyacrylates, pigments, dyes, anticorrosives, neutralizing agents, leveling agents and/or thixotropic agents, suppleness promoters, preservatives, proteins and derivatives thereof, amino acids, vitamins, opacifiers, stabilizers, sequestering agents, complexing agents, pearlescent agents, esthetic enhancers, fatty acids, fatty alcohols, triglycerides, botanical extracts and clarifying agents. The amounts of the various additives are known to the person skilled in the art for the range to be used and are, for example, in the range from 0% to 25% by weight, preferably from 0% to 15% by weight, or preferably from 0.001% to 15% by weight, or preferably for each individual additive within a range from 0.001% to 5% by weight, or in each case preferably from 0.01% to 3% by weight, based on the total weight of the composition.

The cosmetic compositions of the invention may contain one or more emulsifiers or surface-active agents.

Thus, oil-in-water emulsions (O/W) of the invention preferably contain at least one emulsifier having an HLB value>7 and optionally a coemulsifier.

Advantageously, the following nonionic emulsifiers are used:
a) partial fatty acid esters and fatty acid esters of polyhydric alcohols and their ethoxylated derivatives (e.g. glyceryl monostearate, sorbitan stearate, glyceryl stearyl citrate, sucrose stearate)
b) ethoxylated fatty alcohols and fatty acids.

Particularly advantageous nonionic O/W emulsifiers are ethoxylated fatty alcohols or fatty acids, preferably PEG-100 stearate, PEG-40 stearate, ceteareth-20, ceteth-20, steareth-20, ceteareth-12, ceteth-12, steareth-12 and esters of mono-, oligo- or polysaccharides with fatty acids, preferably cetearyl glucoside, methyl glucose distearate.

Advantageous anionic emulsifiers are soaps (e.g. sodium or triethanolamine salts of stearic acid or palmitic acid) and esters of citric acid such as glyceryl stearate citrate.

Suitable coemulsifiers used for the O/W emulsions of the invention may be fatty alcohols having 8 to 30 carbon atoms, monoglycerol esters of saturated or unsaturated, branched or unbranched alkanecarboxylic acids having a chain length of 8 to 24 carbon atoms, especially 12 to 18 carbon atoms, propylene glycol esters of saturated or unsaturated, branched or unbranched alkanecarboxylic acids having a chain length of 8 to 24 carbon atoms, especially 12 to 18 carbon atoms, and sorbitan esters of saturated or unsaturated, branched or unbranched alkanecarboxylic acids having a chain length of 8 to 24 carbon atoms, especially 12 to 18 carbon atoms.

Particularly advantageous coemulsifiers are glyceryl monostearate, glyceryl monooleate, diglyceryl monostearate, sorbitan monoisostearate, sucrose distearate, cetyl alcohol, stearyl alcohol, behenyl alcohol, isobehenyl alcohol, and polyethylene glycol (2) stearyl ether (steareth-2).

It may be advantageous in the context of the present invention to use further emulsifiers. For example, is is possible to further increase the water resistance of the formulations of the invention. Suitable emulsifiers are, for example, alkyl methicone copolyols and alkyl dimethicone copolyols, especially cetyl dimethicone copolyol, lauryl methicone copolyol, W/O emulsifiers such as sorbitan stearate, glyceryl stearate, glycerol stearate, sorbitan oleate, lecithin, glyceryl isostearate, polyglyceryl-3 oleate, polyglyceryl-3 diisostearate, PEG-7 hydrogenated castor oil, polyglyceryl-4-isostearate, acrylate/C₁₀₋₃₀ alkyl acrylate crosspolymer, sorbitan isostearate, Poloxamer 101, polyglyceryl-2 dipolyhydroxystearate, polyglyceryl-3 diisostearate, polyglyceryl-4 dipolyhydroxystearate, PEG-30 dipolyhydroxystearate, diisostearoylpolyglyceryl-3 diisostearate, glycol distearate and polyglyceryl-3 dipolyhydroxystearate.

The cosmetic compositions preferably contain thickeners, especially the water phase, for example in an O/W composition. Advantageous thickeners are:
Crosslinked or non-crosslinked acrylic or methacrylic acid homo- or copolymers. These include crosslinked homopolymers of methacrylic acid or acrylic acid, copolymers of acrylic acid and/or methacrylic acid and monomers derived from other acrylic or vinyl monomers, such as C10-30 alkyl acrylates, C10-30 alkyl methacrylates and vinyl acetate.
Thickening polymers of natural origin, for example based on cellulose, guar gum, xanthan, scleroglucan, gellan gum, rhamsan and karaya gum, alginates, maltodextrin, starch and its derivatives, locust bean gum, hyaluronic acid, carrageenan.
Nonionic, anionic, cationic or amphoteric associative polymers, for example based on polyethylene glycols and their derivatives, or polyurethanes.
Crosslinked or noncrosslinked homopolymers or copolymers based on acrylamide or methacrylamide, such as homopolymers of 2-acrylamido-2-methylpropanesulfonic acid, copolymers of acrylamide or methacrylamide and methacryloyloxyethyltrimethylammonium chloride or copolymers of acrylamide and 2-acrylamido-2-methylpropanesulfonic acid.

Particularly advantageous thickeners are thickening polymers of natural origin, crosslinked acrylic acid or methacrylic acid homo- or copolymers and crosslinked copolymers of 2-acrylamido-2-methylpropanesulfonic acid.

Very particularly advantageous thickeners are xanthan gum, such as the products supplied under the Keltrol® and Kelza® names by CP Kelco or the products from RHODIA with the Rhodopol® name and guar gum, such as those sold under the Jaguar® HP105 name by RHODIA.

Very particularly advantageous thickeners are also crosslinked homopolymers of methacrylic acid or acrylic acid, which are commercially available from Lubrizol under the Carbopol® 940, Carbopol® 941, Carbopol® 980, Carbopol® 981, Carbopol® ETD 2001, Carbopol® EDT 2050, Carbopol® 2984, Carbopol® 5984 and Carbopol® Ultrez 10 names, from 3V under the Synthalen® K, Synthalen® L and Synthalen® MS names, and from PROTEX under the Modarez® V 1250 PX, Modarez® V2000 PX, Viscaron® A1600 PE and Viscaron® A700 PE names.

Very particular advantageous thickeners are crosslinked copolymer of acrylic acid or methacrylic acid and a C₁₀₋₃₀-alkyl acrylate or C₁₀₋₃₀-alkyl methacrylate and copolymers of acrylic acid or methacrylic acid and vinylpyrrolidone. Such polymers are commercially available, for example, from Lubrizol under the Carbopol® 1342, Carbopol® 1382, Pemulen® TR1 or Pemulen® TR2 names and from ISP under the Ultrathix® P-100 (INCI: Acrylic Acid/VP Crosspolymer) names.

Very particularly advantageous thickeners are crosslinked copolymers of 2-acrylamido-2-methylpropanesulfonic acid. Such copolymers are available, for example, from Clariant under the Aristoflex® AVC names (INCI: Ammonium Acryloyldimethyltaurate/VP Copolymer).

If the thickeners are used, the thickeners are generally present in a concentration of about 0% to 2% by weight, preferably 0% to 1% by weight or more preferably within a range from 0.01% to 1% by weight, based on the total weight of the composition of the invention.

The preferred solvents as component K2 are, for example, the aliphatic alcohols having C1-4 carbon atoms, such as ethanol and isopropanol; polyol and its derivatives such as propylene glycol, dipropylene glycol, butylene 1,3-glycol, polypropylene glycol, glycol ethers such as alkyl (C1-4) ethers of mono-, di- or tripropylene glycol or mono-, di- or triethylene glycol, and mixtures thereof.

The proportion of the solvent or solvents in the composition of the invention may, for example, be in the range from 0% to 25% by weight and preferably 0% to 15% by weight, based on the total weight of the composition.

The compositions of the invention may further contain a propellant gas.

Preferred propellants are hydrocarbons such as propane, isobutane and n-butane and mixtures thereof. Compressed air, carbon dioxide, nitrogen, nitrogen dioxide and dimethyl ether and mixtures of all these gases can likewise be used.

Of course, the person skilled in the art is aware that there are propellants that are nontoxic per se and would in principle be suitable for the implementation of the present invention in the form of aerosol preparations, but which should nevertheless be dispensed with because of a critical effect on the environment or other accompanying circumstances. These are especially fluorocarbons and chlorofluorocarbons (CFCs) such as 1,2-difluoroethane (propellant 152 A).

Active haircare ingredients are preferably included in the cosmetic composition. Care substances used with preference may be cyclic polydimethylsiloxanes (cyclomethicones) or silicone surfactants (polyether-modified siloxanes) of the dimethicone copolyol or simethicone type. Cyclomethicones are supplied, inter alia, under the Abil® K4 trade names by Goldschmidt or, for example, DC 244, DC 245 and DC 345 by Dow Corning. Dimethicone copolyols are supplied, for example, under the DC 193 trade name by Dow Corning or Belsil® DM 6031 by Wacker.

Surfactants

The compositions of the invention may also contain surfactants selected from the group of anionic, cationic, nonionic and/or amphoteric surfactants.

Advantageous anionic surfactants in the context of the present invention are:
acylamino acids and their salts, such as acylglutamates, especially sodium acylglutamate, and sarcosinates, for example myristoylsarcosine, TEA-lauroyl sarcosinate, sodium lauroylsarcosinate and sodium cocoylsarcosinate,
sulfonic acids and salts thereof, such as acyl isethionate, for example sodium or ammonium cocoylisethionate, sulfosuccinates, for example sodium dioctyl sulfosuccinate, disodium laureth sulfosuccinate, disodium lauryl sulfosuccinate and disodium undecyleneamido MEA sulfosuccinate, disodium PEG-5 lauryl citrate sulfosuccinate and derivatives;
sulfuric esters, such as alkyl ether sulfate, for example sodium, ammonium, magnesium, MIPA and TIPA laureth sulfate, sodium myrethsulfate and sodium C₁₂ to C₁₃-parethsulfate, and alkyl sulfates, for example sodium, ammonium and TEA lauryl sulfate,
taurates, for example, sodium lauroyl taurate and sodium methyl cocoyl taurate;
ether carboxylic acids, for example sodium laureth-13 carboxylate and sodium PEG-6 cocamide carboxylate, sodium PEG-7 olive oil carboxylate;
phosphoric esters and salts, such as DEA-oleth-10 phosphate and dilaureth-4 phosphate;
alkylsulfonates, for example sodium cocomonoglyceride sulfate, sodium C₁₂ to C₁₄ olefinsulfonate, sodium lauryl sulfoacetate and magnesium PEG-3 cocamide sulfate,
acylglutamates, such as di-TEA-palmitoylaspartate and sodium caprylic/capric glutamate;
acyl peptides, for example, palmitoyl hydrolyzed milk protein, sodium cocoyl hydrolyzed soy protein, and sodium/potassium cocoyl hydrolyzed collagen;
carboxylic acids and derivatives, for example lauric acid, aluminum stearate, magnesium alkanoate and zinc undecylenoate, ester carboxylic acids, for example, calcium stearoyl lactylate, laureth-6 citrate, and sodium PEG-4 lauramide carboxylate;
alkylarylsulfonates.

Advantageous cationic surfactants in the context of the present invention are quaternary surfactants. Quaternary surfactants contain at least one nitrogen atom covalently bonded to 4 alkyl or aryl groups. Advantageous examples include alkyl betaine, alkylamidopropyl betaine and alkylamidopropyl hydroxysultaine.

Further advantageous cationic surfactants for the purposes of the present invention are also alkylamines, alkylimidazoles and ethoxylated amines and especially their salts.

Advantageous amphoteric surfactants for the purposes of the present invention are acyl/dialkylethylenediamines, for example sodium acylamphoacetate, disodium acylamphodipropionate, disodiumalkylamphodiacetate, sodiumacylamphohydroxypropylsulfonate, disodium acylamphodiacetate, sodium acylamphopropionate, and N-coconut fatty acid amidoethyl-N-hydroxyethylglycinate sodium salts.

Further advantageous amphoteric surfactants are N-alkylamino acids, for example aminopropylalkylglutamide, alkylaminopropionic acid, sodium alkylimidodipropionate and lauroamphocarboxyglycinate.

Advantageous active nonionic surfactants for the purposes of the present invention are alkanolamides, such as cocamide MEA/DEA/MIPA, esters formed by esterification of carboxylic acids with ethylene oxide, glycerol, sorbitan or other alcohols, ethers, for example ethoxylated alcohols, ethoxylated lanolin, ethoxylated polysiloxanes, propoxylated POE ethers, alkyl polyglycosides, such as lauryl glucoside, decyl glycoside and cocoglycoside, glycosides with an HLB value of at least 20 (e.g. Belsil SPG® 128V from Wacker).

Further advantageous nonionic surfactants are alcohols and amine oxides, such as cocoamidopropylamine oxide.

Among the alkyl ether sulfates, sodium alkyl ether sulfates based on di- or triethoxylated lauryl and myristyl alcohol are especially preferred. They clearly surpass the alkyl sulfates with regard to insensitivity to water hardness, thickenability, cold solubility and, in particular, skin and mucous membrane compatibility. Lauryl ether sulfate has better foaming properties than myristyl ether sulfate, but is inferior in terms of mildness.

Alkyl ether carboxylates with moderate and especially with higher are among the mildest surfactants that exist, but have poor foaming and viscosity characteristics. They are often used in combination with alkyl ether sulfates and amphoteric surfactants.

Sulfosuccinic esters (sulfosuccinates) are mild and readily foaming surfactants but are preferably used only together with other anionic and amphoteric surfactants on account of their poor thickenability, and only in neutral or well-buffered products on account of their low hydrolysis stability.

Amidopropyl betaines have excellent skin and eye mucous membrane compatibility. In combination with anionic surfactants, their mildness can be synergistically improved. Preference is given to the use of cocamidopropyl betaine.

Amphoacetates/amphodiacetates, being amphoteric surfactants, have very good skin and mucous membrane compatibility and can have a conditioning effect or increase the care effect of additives. They are used in a similar manner to the betaines for optimization of alkyl ether sulfate formulations. Most preferred are sodium cocoamphoacetate and disodium cocoamphodiacetate.

Alkyl polyglycosides are mild and have good universal properties, but poor foaming. For this reason, they are preferably used in combination with anionic surfactants.

Conditioners

Optionally, the compositions of the invention contain a conditioner. Preferred conditioners are, for example, all compounds listed in the International Cosmetic Ingredient Dictionary and Handbook (Volume 4, publisher: R. C. Pepe, J. A. Wenninger, G. N. McEwen, The Cosmetic, Toiletry, and Fragrance Association, 9th Edition, 2002) in Section 4 under the headings of Hair Conditioning Agents, Humectants, Skin-Conditioning Agents, Skin-Conditioning Agents-Emollient, Skin-Conditioning Agents-Humectant, Skin-Conditioning Agents-Miscellaneous, Skin-Conditioning Agents-Occlusive and Skin Protectants, and all compounds listed in EP-A 934 956 (p. 11-13) under “water soluble conditioning agent” and “oil soluble conditioning agent”.

Particular advantageous conditioners are, for example, the compounds referred to as Polyquaternium according to INCI (especially Polyquaternium-1 to Polyquaternium-114).

Suitable conditioners include, for example, polymeric quaternary ammonium compounds, cationic cellulose derivatives, chitosan derivatives, guar gum derivatives and polysaccharides, especially guar hydroxypropylammonium chloride (e.g. Jaguar® Excel, Jaguar® 162 from Rhodia).

Further conditioners that are advantageous in accordance with the invention are nonionic poly-N-vinylpyrrolidone/polyvinyl acetate copolymers (e.g. Luviskol®VA 64 from BASF AG), anionic acrylate copolymers (e.g. Luviflex®Soft from BASF AG), and/or amphoteric amide/acrylate/methacrylate copolymers (e.g. Amphomer® from National Starch). Further possible conditioners are quaternized silicones.

Optionally, conventional additives may likewise be included in the composition, for example to impart certain modifying properties thereto. These may be, for instance, silicones or silicone derivatives, wetting agents, humectants, plasticizers such as glycerol, glycol and phthalic esters and ethers, odorants and perfumes, UV absorbers, dyes, pigments and other colorants, anticorrosive agents, neutralizing agents, antioxidants, antisticking agents, combining agents and conditioners, antistats, shine agents, preservatives, proteins and derivatives thereof, amino acids, vitamins, emulsifiers, surface-active agents, viscosity modifiers, thickeners and rheology modifiers, gelling agents, opacifiers, stabilizers, surfactants, sequestrants, complexing agents, pearlescent agents, esthetic enhancers, fatty acids, fatty alcohols, triglycerides, botanical extracts, clarifying aids and film formers.

These additives are generally present in a concentration of about 0.001% to 15% by weight, preferably 0.01% to 10% by weight, based on the total weight of the composition.

In a preferred embodiment of the hair cosmetics composition, at least one film former of component K3 is selected from the group consisting of a nonionic polymer, an anionic polymer, an amphoteric polymer and/or a cationic polymer.

Preferably, the film former K3 is selected from the group consisting of a polyacrylate, a polyacrylamide, a polyurethane, a polyurea, a polysiloxane or a mixture of at least two of these. The polyacrylates also include silicone-acrylate copolymers.

To improve the resistance of decorative products to water, tears or sweat (often called water resistance) among other reasons, film-forming polymers are used as film formers.

Preferred film-forming polymers selected are polymers based on acrylates or vinylpyrrolidones. Advantageous film formers are trimethylsiloxysilicates, silicone acrylate copolymers (e.g. TIB4-200 from Dow Corning or KP-561 from Shin Etsu), trimethyl pentaphenyl trisiloxanes (Dow Corning 555 Cosmetic Fluid from Dow Corning Ltd.) or vinylpyrrolidone copolymer (e.g. PVP/eicosene copolymer or PVP/hexadecane copolymer).

Preference is generally given to using nonionic, anionic, amphoteric and/or cationic polymers as film formers. Preferably nonionic polymers that are used in the cosmetic composition, either alone or in combination, preferably with anionic and/or amphoteric and/or zwitterionic polymers, are preferably selected from the group consisting of:
polyalkyloxazolines;
vinyl acetate homopolymers or copolymers, including, for example, copolymers of vinyl acetate and acrylic esters, copolymers of vinyl acetate and ethylene, copolymers of vinyl acetate and maleic esters;
acrylic ester copolymers, for example the copolymers of alkyl acrylate and alkyl methacrylate, copolymers of alkyl acrylate and urethanes;
copolymers of acrylonitrile and nonionic monomer selected from butadiene and (meth)acrylate;
styrene homo- and copolymers, including, for example, homopolystyrene, copolymers of styrene and alkyl (meth)acrylate, copolymers of styrene, alkyl methacrylate and alkyl acrylate, copolymers of styrene and butadiene, copolymers of styrene, butadiene and vinylpyridine;
polyamides;
vinyllactam homo- or copolymers, such as vinylpyrrolidone homo- or copolymers, including, for example, polyvinylpyrrolidone, polyvinylcaprolactam, copolymers of N-vinylpyrrolidone and vinyl acetate and/or vinyl propionate in various concentration ratios, polyvinylcaprolactam, polyvinylamides and salts thereof, and also copolymers of vinylpyrrolidone and dimethylaminoethyl methacrylate, terpolymers of vinylcaprolactam, vinylpyrrolidone and dimethylaminoethyl methacrylate;
polysiloxanes;
polyimides such as Aqua Flex™ XL-30 from Ashland;
homopolymers of N-vinylformamide such as PVF from AkzoNobel; and
polyurethanes such as Baycusan® C2000 from Covestro Deutschland AG.

Particular preferred nonionic polymers are acrylic ester copolymers, homopolymers of vinylpyrrolidone and copolymers, and polyvinylcaprolactam.

Particularly preferred nonionic polymers are homopolymers of vinylpyrrolidone, e.g. Luviskol® K from BASF, copolymers of vinylpyrrolidone and vinyl acetate, e.g. Luviskol® VA products from BASF or PVPVA® S630L from ISP, terpolymers of vinylpyrrolidone, vinyl acetate and propionate, e.g. Luviskol® VAP from BASF, and polyvinylcaprolactams, e.g. Luviskol® PLUS from BASF.

Advantageous anionic polymers are homo- or copolymers with monomer units which contain acid groups and have optionally been copolymerized with comonomers not containing acid groups. Suitable monomers are unsaturated, free-radically polymerizable compounds having at least one acid group, and especially carboxylic acid, sulfonic acid or phosphonic acid.

Examples of anionic polymers containing carboxylic acid groups are:
acrylic acid or methacrylic acid homo- or copolymers or salts thereof. These include, for example, the copolymers of acrylic acid and acrylamide and/or sodium salts thereof, copolymers of acrylic acid and/or methacrylic acid and an unsaturated monomer selected from the group of ethylene, styrene, vinyl esters, acrylic esters, methacrylic esters, optionally ethoxylated compounds, copolymers of vinylpyrrolidone, acrylic acid and C1-C20 alkyl methacrylates, e.g. Acrylidone® LM from ISP, copolymers of methacrylic acid, ethyl acrylate and tert-butyl acrylate, e.g. Luvimer® 100 P from BASF;
crotonic acid derivative homo- or copolymers or salts thereof. These include, for example, vinyl acetate/crotonic acid, vinyl acetate/acrylate and/or vinyl acetate/vinyl neodecanoate/crotonic acid copolymers, for example Resyn® 28-1310 or Resyn® 28-2930 from AkzoNobel or Luviset® CAN from BASF, sodium acrylate/vinyl alcohol copolymers;
unsaturated C4-C8 carboxylic acid derivatives or carboxylic anhydride copolymers selected from copolymers of maleic acid/maleic anhydride or fumaric acid/fumaric anhydride or itaconic acid/itaconic anhydride and at least one monomer selected from the group of vinyl esters, vinyl ethers, vinyl halogen derivatives, phenyl vinyl derivatives, acrylic acid, acrylic esters or copolymers of maleic acid/maleic anhydride or fumaric acid/fumaric anhydride or itaconic acid/itaconic anhydride and at least one monomer selected from the group of allyl esters, methallyl esters and optionally acrylamides, methacrylamides, alpha-olefin, acrylic esters, methacrylic esters, vinylpyrrolidones. Further preferred polymers are methyl vinyl ethers/maleic acid copolymers obtained through hydrolysis of vinyl ether/maleic anhydride copolymers. These polymers may also be partly esterified (ethyl, isopropyl or butyl esters) or partly amidated;
water-soluble or -dispersible anionic polyurethanes, e.g. Luviset.® PUR from BASF, and DynamX® from AkzoNobel, Baycusan® C1000, Baycusan® C1001, Baycusan® C1003, Baycusan® C1004, Baycusan® C1008 from Covestro Deutschland AG.

This preceding list should be considered nonexhaustive and hence nonlimiting.

Advantageous anionic polymers containing sulfonic acid group are salts of polyvinylsulfonic acids, polystyrenesulfonic acids, for example sodium polystyrenesulfonate, or polyacrylamidosulfonic acids.

Particularly advantageous anionic polymers are acrylic acid copolymers, crotonic acid derivative copolymer, copolymers of maleic acid/maleic anhydride or fumaric acid/fumaric anhydride or itaconic acid/itaconic anhydride and at least one monomer selected from the group of vinyl esters, vinyl ethers, vinyl halogen derivatives, phenyl vinyl derivatives, acrylic acid, acrylic esters and salts of polystyrenesulfonic acids.

Very particularly advantageous anionic polymers are acrylate copolymers, for example Luvimer® from BASF, ULTRAHOLD® STRONG ethyl acrylate/N-tert-butylacrylamide/acrylic acid copolymers from BASF, VA/crotonate/vinyl neodecanoate copolymer, for example Resyn® 28-2930 from AkzoNobel, copolymers, for example copolymers of methyl vinyl ether and maleic anhydride in partly esterified form, e.g. GANTREZ® from Ashland, and sodium polystyrenesulfonates, such as Flexan® 130 from AkzoNobel.

Advantageous amphoteric polymers may be selected from the polymers containing units A and B distributed randomly in the polymer chain, where A is a unit derived from a monomer having at least one basic nitrogen atom, and B represents a unit that originates from an acidic monomer having one or more carboxyl or sulfo groups. Alternatively, A and B may be groups derived from zwitterionic carboxybetaine monomers or sulfobetaine monomers. A and B may also be a cationic polymer chain containing primary, secondary, tertiary or quaternary groups, where at least one amino group bears a carboxyl group or sulfo group bonded via a hydrocarbyl group, or A and B are part of a polymer chain with an ethylene-α,β-dicarboxylic unit in which the carboxylic acid groups have been reacted with a polyamine containing one or more primary or secondary amino groups.

Preferred amphoteric polymers are:
polymers which are formed in the copolymerization of a monomer derived from a vinyl compound having a carboxyl group, such as, more particularly, acrylic acid, methacrylic acid, maleic acid, α-chloroacrylic acid, and a basic monomer. The basic monomer is derived from a vinyl compound which is substituted and contains at least one basic atom, such as, more particularly, dialkylaminoalkyl methacrylate and acrylate, dialkylaminoalkylmethacrylamide and -acrylamide. Compounds of this kind have been described in American patent U.S. Pat. No. 3,836,537.
Polymers having units derived from: a) at least one monomer selected from the acrylamides or methacrylamides, substituted by an alkyl group on the nitrogen atom, b) at least one acidic comonomer containing one or more reactive carboxyl groups, and c) at least one basic comonomer, such as esters of acrylic acid and methacrylic acid with primary, secondary, tertiary and quaternary amino substituents and the quaternization product of dimethylaminoethyl methacrylate with dimethyl sulfate or diethyl sulfate.

Particularly preferred N-substituted acrylamides or methacrylamides are compounds wherein the alkyl groups contain 2 to 12 carbon atoms. Very particular preference is given to N-ethylacrylamide, N-t-butylacrylamide, N-t-octylacrylamide, N-octylacrylamide, N-decylacrylamide, N-dodecylacrylamide and the corresponding methacrylamides.

Suitable acidic comonomers are especially selected from the group of acrylic acid, methacrylic acid, crotonic acid, itaconic acid, maleic acid, fumaric acid and the alkyl monoesters having 1 to 4 carbon atoms of maleic acid, maleic anhydride, fumaric acid or fumaric anhydride.

Preferred basic comonomers are aminoethyl methacrylate, butylaminoethyl methacrylate, N,N-dimethylaminoethyl methacrylate, N-t-butylaminoethyl methacrylate.
Crosslinked and wholly or partly acylated polyaminoamides derived from polyaminoamides of the following general formula:

—[CO—R—CO—Z]—

where R is a divalent group derived from a saturated dicarboxylic acid, an aliphatic mono- or dicarboxylic acid having an ethylenic double bond, an ester of these acids with a lower alkanol having 1 to 6 carbon atoms, or a group which forms in the addition of one of these acids onto a bis-primary or bis-secondary amine, and Z is a group derived from a bis-primary, mono- or bis-secondary polyalkylenepolyamine, and preferably: a) as a quantitative fraction of 60 to 100 mol % the —NH—[(CH₂)ₓ—NH-]p- groups where x=2 and p=2 or 3 or x=3 and p=2, the group being formed by diethylenetriamine, triethylenetetramine or dipropylenetriamine; b) as a quantitative fraction of 0 to 40 mol % the —NH—[(CH₂)ₓ-NH-]p- group where x=2 and p=1, which is obtainable from ethylenediamine, or the group that originates from piperazine:

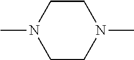

c) in a quantitative fraction of 0 to 20 mol % the —H—(CH₂)₆—NH— group obtainable from hexamethylenediamine, these polyaminoamides being crosslinked by addition of a bifunctional crosslinker, preferably selected from the group of epihalohydrins, diepoxides, dianhydrides and bis-unsaturated derivatives, in an amount within a range from 0.025 to 0.35 mol of crosslinker per amino group of the polyaminoamide, and acylated with acrylic acid, chloroacetic acid or an alkanesulfone or salts thereof.

The saturated carboxylic acids are preferably from the acids having 6 to 10 carbon atoms, such as adipic acid, 2,2,4-trimethyladipic acid and 2,4,4-trimethyladipic acid, terephthalic acid; acids having an ethylenic double bond, for example acrylic acid, The alkane sultones used in the acylation are preferably propane sultone or butane sultone; the salts of the acylating agents are preferably the sodium salts or potassium salts.
Polymers having zwitterionic units of the following formula:

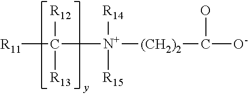

in which R₁₁ is a polymerizable unsaturated group, such as acrylate, methacrylate, acrylamide or methacrylamide, y and z are integers from 1 to 3, R₁₂ and R₁₃ are a hydrogen atom, methyl, ethyl or propyl, R₁₄ and R₁₅ are a hydrogen atom or an alkyl group chosen such that the sum total of the carbon atoms R₁₄ and R₁₅ does not exceed 10.

Polymers containing such units may also have units that originate from non-zwitterionic monomers, such as dimethyl- and diethylaminoethyl acrylate or dimethyl and diethylaminoethyl methacrylate or alkyl acrylates or alkyl methacrylates, acrylamides or methacrylamides or vinyl acetate.
Polymers which are derived from chitosan and contain monomer units conforming to the following formulae:

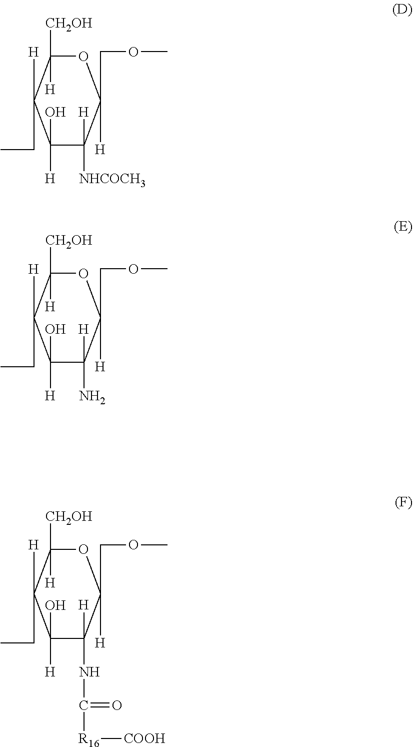

where the first unit is present in proportions of 0% to 30%, the second unit in proportions of 5% to 50% and the third unit in proportions of 30% to 90%, with the proviso that R₁₆ in the third unit is a group of the following formula:

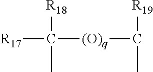

in which, if q=0, the R₁₇, R₁₈ and R₁₉ groups are the same or different and are each a hydrogen atom, methyl, hydroxyl, acetoxy or amino, a monoalkylamine radical or a dialkylamine radical which is optionally interrupted by one or more nitrogen atoms and/or optionally by one or more amino groups, hydroxyl groups, carboxyl groups, alkylthio groups, sulfo groups, alkylthio groups wherein the alkyl groups bears an amino radical, where at least one of the R₁₇, R₁₈ and R₁₉ groups in this case is a hydrogen atom; or, if q=1, the R₁₇, R₁₈, and R₁₉ groups are each a hydrogen atom, and the salts that form these compounds with bases or acids.
Polymers which conform to the following general formula and are described, for example, in French Patent 1 400 366:

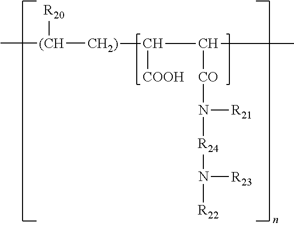

in which R₂₀ is a hydrogen atom, CH₃O, CH₃CH₂ or phenyl, R₂₁ is a hydrogen atom or a lower alkyl group, such as methyl or ethyl, R₂₂ is a hydrogen atom or a lower C₁₋₆-alkyl group, such as methyl or ethyl, R₂₃ is a lower C₁₋₆-alkyl group, such as methyl or ethyl, or a group of the formula: —R₂₄—N(R₂₂)₂ where R₂₄ is a —CH₂—CH₂, —CH₂—CH₂—CH₂— or —CH₂—CH(CH₃)— group and where R₂₂ has the definitions given above.
Polymers which can be formed in the N-carboxyalkylation of chitosan, such as N-carboxymethylchitosan or N-carboxybutylchitosan.
Alkyl(C₁₋₅) vinyl ether/maleic anhydride copolymers which have been partly modified partly by semiamidation with an N,N-dialkylaminoalkylamine such as N,N-dimethylaminopropylamine or an N,N-dialkylamino alcohol. These polymers may also contain further comonomers, such as vinylcaprolactam.

Very particularly advantageous amphoteric polymers are the copolymers octylacrylamide/acrylates/butylaminoethyl methacrylate copolymer, which are sold under the AMPHOMER®, AMPHOMER® LV 71 or BALANCE® 47 names by AkzoNobel, and methyl methacrylate/methyl dimethylcarboxymethylammonioethylmethacrylate copolymers.

It may be advantageous to neutralize the anionic and amphoteric polymers with suitable bases to improve their water solubility or their water dispersibility.

Neutralizing agents used for polymers containing acid groups may be the following bases: Hydroxides wherein the cation is ammonium or an alkali metal, for example NaOH or KOH.

Other neutralizing agents are primary, secondary or tertiary amines, amino alcohols or ammonia. Preference is given here to using 2-amino-2-methylpropane-1,3-diol (AMPD), 2-amino-2-ethylpropane-1,3-diol (AEPD), 2-amino-2-methyl-1-propanol (AMP), 2-amino-i-butanol (AB), 2-aminopropane-1,3-diol, monoethanolamine (MEA), diethanolamine (DEA), triethanolamine (TEA), monoisopropanolamine (MIPA), diisopropanolamine (DIPA), triisopropanolamine (TIPA), Dimethyl Laurylamine (DML), Dimethyl Myristalamine (DMM), and Dimethyl Stearamine (DMS).

The neutralization may be partial or complete according to the end use.

It is optionally possible to use, albeit less preferably, cationic polymers, for example polymers containing primary, secondary, tertiary and/or quaternary amino groups that are part of the polymer chain or bonded directly to the polymer chain.

[A6] In a preferred embodiment of the hair cosmetics composition, the film former K3 is a polyurethane obtainable by reacting one or more water-insoluble, non-water-dispersible, isocyanate-functional polyurethane prepolymers A) with one or more amino-functional compounds B).

In the context of the invention, the term “water-insoluble, non-water-dispersible polyurethane prepolymer” means more particularly that the water solubility of the prepolymer used in accordance with the invention at 23° C. is less than 10 g/liter, preferably less than 5 g/liter, and the prepolymer at 23° does not result in a sedimentation-stable dispersion in water, especially deionized water. In other words, the prepolymer settles out when an attempt is made to disperse it in water.

Preferably, the NCO-terminated polyurethane prepolymer A) is obtainable from the reaction of a reaction mixture comprising a polyisocyanate and polyol. The polyisocyanate preferably has a functionality within a range from >1.5 to 6, or preferably from 1.8 to 5, or preferably from 2 to 4, especially of 2. Suitable polyisocyanates are aliphatic, aromatic araliphatic or cycloaliphatic polyisocyanates. Examples of such suitable polyisocyanates are butylene 1,4-diisocyanate, hexamethylene 1,6-diisocyanate (HDI), isophorone diisocyanate (IPDI), 2,2,4- and/or 2,4,4-trimethylhexamethylene diisocyanate, the isomeric bis(4,4′-isocyanatocyclohexyl)methanes or mixtures thereof of any isomer content, cyclohexylene 1,4-diisocyanate, 4-isocyanatomethyloctane 1,8-diisocyanate (nonane triisocyanate), phenylene 1,4-diisocyanate, toluene 2,4- and/or 2,6-diisocyanate, naphthylene 1,5-diisocyanate, diphenylmethane 2,2′- and/or 2,4′- and/or 4,4′-diisocyanate, 1,3- and/or 1,4-bis(2-isocyanatoprop-2-yl)benzene (TMXDI), 1,3-bis(isocyanatomethyl)benzene (XDI) and alkyl 2,6-diisocyanatohexanoate (lysine diisocyanates) with C1 to C8 alkyl groups.

Preferably, the polyisocyanate is an aliphatic polyisocyanate. Preferred aliphatic diisocyanates are hexamethylene diisocyanate and isophorone diisocyanate and mixtures thereof.

The polyol preferably has a functionality of >1.5 to 6 or preferably from 1.8 to 5, or preferably from 2 to 4, especially of 2. Preferably, the polyol is selected from the group consisting of a polyether polyol, a polycarbonate polyol, a polyether polycarbonate polyol, a polyester polyol or a mixture of at least two of these. Preferably, the polyol includes a polyol containing polyoxyethylene groups. With regard to the polyols, preference is given to copolymers of ethylene oxide and propylene oxide having an ethylene oxide content, based on the total amount of the oxyalkylene groups present, of 60 to 85 mol %. Preference is also given to polyesterpolyols preferably formed from an acid selected from the group consisting of malonic acid, succinic acid, glutaric acid, adipic acid, pimelic acid, suberic acid, azelaic acid, sebacic acid or a mixture of at least two of these with a polyol selected from the group consisting of ethane-1,2-diol, propane-1,3-diol, 2,2-dimethylpropane-1,3-diol (neopentanediol), butane-1,4-diol, 2,2-dimethylbutane-1,4-diol, pentane-1,5-diol, 2,2-dimethylbutane-1,4-diol, hexane-1,6-diol or a mixture of at least two of these.

In a preferred embodiment of the hair cosmetics composition, the total amount of carbodiimides and the total amount of film formers is present in a ratio of 2:1 to 1:4, or preferably in a ratio of 1.5:1 to 1:3, or preferably in a ratio from 1:1 to 1:3. The cosmetic composition preferably includes a higher percentage by weight of film formers than of compounds containing at least three carbodiimide groups, also called polycarbodiimide.

In a preferred embodiment of the hair cosmetics composition, the composition is selected from the group consisting of a pump spray, an aerosol, a gel, a foam, a mousse, a lotion, a wax, a pomade, an oil, a milk, an oil-in-water emulsion, an aqueous solution or a cream. The cosmetic composition is preferably in the form of a pump spray, an aerosol, a gel, a foam, a mousse, a lotion, a wax, a pomade.

The composition of the invention may further comprise a wax.

In the context of the present document, a wax is defined as a lipophilic fatty substance which is solid at room temperature (25° C.) and shows a reversible solid/liquid change of state at a melting temperature between 30° C. and 200° C. Above the melting point, the viscosity of the wax is low and it becomes miscible with oils.

The wax is advantageously chosen from the groups of natural waxes, for example cotton wax, carnauba wax, candelilla wax, esparto wax, japan wax, montan wax, sugarcane wax, beeswax, wool wax, shellac, microwaxes, ceresin, ozokerite, ouricury wax, cork fiber wax, lignite waxes, berry wax, shea butter, or synthetic waxes such as paraffin waxes, polyethylene waxes, waxes produced by Fischer-Tropsch synthesis, hydrogenated oils, fatty acid esters and glycerides that are solid at 25° C., silicone waxes and derivatives (alkyl derivatives, alkoxy derivatives and/or esters of polymethylsiloxane) and mixtures thereof. The waxes may take the form of stable dispersions of colloidal wax particles which can be produced by known methods, for example according to “Microemulsions Theory and Practice”. L. M. Prince Ed., Academic Press (1977), pages 21-32.

The waxes may be present in amounts of 0% to 10% by weight, based on the total weight of the composition, and preferably 0% to 5% by weight.

The preferred cosmetically acceptable medium of the composition of the invention comprises water and optionally a cosmetically acceptable water-miscible suitable organic solvent.

The water used in the composition according to the invention may be a blossom water, pure demineralized water, mineral water, thermal water and/or seawater.

In the case of an O/W composition as the composition of the invention, the water content may be in the range from 40% to 95% by weight, preferably in the range from 50% to 90% by weight, most preferably in the range from 60% to 80% by weight, based on the total weight of the composition. In the case of a W/O composition, the water content is in the range from 0% to 60% by weight, preferably in the range from 10% to 50% by weight, most preferably in the range from 30% to 50% by weight, based on the total weight of the composition.

The composition may also be foamed with a propellant gas. The above-described emulsions may be stabilized by O/W, W/O or W/Si emulsifier, thickener (such as hydrodispersion) or solids (for example a Pickering emulsion).

The composition may contain one or more emulsifiers or surfactants.

Thus, especially oil-in-water emulsions (O/W) preferably contain at least one emulsifier having an HLB value>7 and optionally a coemulsifier.

Advantageously, the following nonionic emulsifiers are used:
a) partial fatty acid esters and fatty acid esters of polyhydric alcohols and their ethoxylated derivatives (e.g. glyceryl monostearate, sorbitan stearate, glyceryl stearyl citrate, sucrose stearate)
b) ethoxylated fatty alcohols and fatty acids.

Particularly advantageous nonionic O/W emulsifiers are ethoxylated fatty alcohols or fatty acids, preferably PEG-100 stearate, PEG-40 stearate, ceteareth-20, ceteth-20, steareth-20, ceteareth-12, ceteth-12, steareth-12 and esters of mono-, oligo- or polysaccharides with fatty acids, preferably cetearyl glucoside, methyl glucose distearate.

Advantageous anionic emulsifiers are soaps (e.g. sodium or triethanolamine salts of stearic acid or palmitic acid) and esters of citric acid such as glyceryl stearate citrate.

Suitable coemulsifiers used for the O/W emulsions of the invention may be fatty alcohols having 8 to 30 carbon atoms, monoglycerol esters of saturated or unsaturated, branched or unbranched alkanecarboxylic acids having a chain length of 8 to 24 carbon atoms, especially 12 to 18 carbon atoms, propylene glycol esters of saturated or unsaturated, branched or unbranched alkanecarboxylic acids having a chain length of 8 to 24 carbon atoms, especially 12 to 18 carbon atoms, and sorbitan esters of saturated or unsaturated, branched or unbranched alkanecarboxylic acids having a chain length of 8 to 24 carbon atoms, especially 12 to 18 carbon atoms.

Particularly advantageous coemulsifiers are glyceryl monostearate, glyceryl monooleate, diglyceryl monostearate, sorbitan monoisostearate, sucrose distearate, cetyl alcohol, stearyl alcohol, behenyl alcohol, isobehenyl alcohol, and polyethylene glycol (2) stearyl ether (steareth-2).

It may be advantageous in the context of the present invention to use further emulsifiers. For example, is is possible to further increase the water resistance of the formulations of the invention. Suitable emulsifiers are, for example, alkyl methicone copolyols and alkyl dimethicone copolyols, especially cetyl dimethicone copolyol, lauryl methicone copolyol, W/O emulsifiers such as sorbitan stearate, glyceryl stearate, glycerol stearate, sorbitan oleate, lecithin, glyceryl isostearate, polyglyceryl-3 oleate, polyglyceryl-3 diisostearate, PEG-7 hydrogenated castor oil, polyglyceryl-4 isostearate, acrylate/Co-so alkyl acrylate crosspolymer, sorbitan isostearate, Poloxamer 101, polyglyceryl-2 dipolyhydroxystearate, polyglyceryl-3 diisostearate, polyglyceryl-4 dipolyhydroxystearate, PEG-30 dipolyhydroxystearate, diisostearoylpolyglyceryl-3 diisostearate, glycol distearate and polyglyceryl-3 dipolyhydroxystearate.

In a preferred embodiment of the hair cosmetics composition, the composition is suitable for treatment of hair, especially for conserving the structure of the change in color and/or shape of the hair. In particular, the treatment of the hair with the hair cosmetics composition constitutes a further hair cosmetics application. The further hair cosmetics application may be any application to the hair that leads to a change in the color and/or shape of the hair. What is achieved by the treatment of the hair with the composition of the invention is preferably that the hair has higher structural resistance to water, for example in the form of a jet of water, for example when showering, bathing or swimming, or haircare products such as shampoo, hair fixatives, etc., low swelling and good tactile properties. Preferably, the hair cosmetics application is selected from the group consisting of
(A) dyeing at least a part of the hair,
(B) a permanent wave treatment of the hair,
(C) a treatment with a curling iron,
(D) straightening of the hair,
(E) permanent straightening of the hair,
(F) dressing of the hair,
(G) hair extension,
(H) hair shortening,
(I) a combination of at least two applications selected from (A) to (H).

The treatment of hair with the hair cosmetics composition of the invention preferably takes place with employment of elevated temperature. The employment of elevated temperature may relate either to the hair to be treated or to the hair cosmetics composition to be used, or to both. Preferably, the employment of elevated temperature selected from the group consisting of heating of the hair cosmetics composition before application on the hair, heating of the hair before treatment with the hair cosmetics composition, heating of the hair after treatment with the hair cosmetics composition, heating of the hair during the treatment with the hair cosmetics composition or a combination of at least two of these takes place. Preferably, the employment of elevated temperature takes place by heating the hair cosmetics composition before treatment of the hair with the hair cosmetics composition. The employment of elevated temperature takes place either before, during and/or after the treatment of hair with the hair cosmetics composition of the invention. Preferably, the hair to be treated, on employment of elevated temperature, is contacted with a composition having a temperature within a range from 30 to 250° C., or preferably within a range from 40 to 230° C., or preferably within a range from 50 to 200° C., or preferably within a range from 60 to 180° C. The heating of the hair cosmetics composition preferably takes place before treatment of the hair therewith, preferably within a range from 40 to 120° C., or preferably within a range from 50 to 110° C., or preferably within a range from 60 to 100° C. Preferably, the elevated temperature is imparted to the hair via heated air or a heated surface. The elevation of the temperature is preferably achieved by a means selected from the group consisting of a hairdryer, a straightener, a curling iron, a hood dryer, a curling wand, or a combination of at least two of these. The specified temperature ranges for the elevated temperature is preferably the temperature of the means immediately before it is brought into contact with the hair.

The duration of the employment of elevated temperature is preferably within a range from 1 minute to 3 hours, or preferably within a range from 5 minutes to 2 hours, or preferably within a range from 10 minutes to 1 hour, or preferably within a range from 20 minutes to 50 minutes.

The invention further relates to use of a composition comprising at least one carbodiimide of the formula ( )

R⁴— [R¹—N═C═N]ₙ—R⁵ (I)

where

n is an integer from 3 to 100, preferably from 3 to 50, or preferably from 3 to 20, or preferably from 3 to 20,

R¹, R⁴ and R⁵ are each independently linear or branched, aliphatic or cycloaliphatic, optionally substituted groups having 1 to 100 carbon atoms, preferably 1 to 50, or preferably 1 to 20,

for fixing structures in hair.

All statements relating to the compound containing at least one carbodiimide, also called polycarbodiimide, which were made in connection with the cosmetic composition of the invention comprising the compound containing at least one carbodiimide are preferably applicable correspondingly to the use for fixing structures in hair. The expression “fixing structures in the hair” shall be understood to mean the fixing of artificial structures and also of endogenous structures. Examples of artificial structures are non-endogenous or artificial hair, or other artificial elements that can be applied to the hair. Examples of endogenous structures are structures that are already present on the user's body, but have been modified in terms of their shape, position or design and are to be fixed, for example hair tresses or parts of a hair tress. Fixing shall be understood in particular to mean that the desired shape is fixed, i.e. is to be conserved over a prolonged period. The use of the hair cosmetics composition of the invention preferably takes place with employment of elevated temperature. The employment of elevated temperature takes place either before, during and/or after the treatment of hair with the hair cosmetics composition of the invention. Preferably, the hair or the hair cosmetics composition to be treated, before, on and/or after the application of the hair cosmetics composition, is brought to a temperature within a range from 30 to 250° C., or preferably within a range from 40 to 230° C., or preferably within a range from 50 to 200° C., or preferably within a range from 60 to 180° C. Preference is given to heating the hair cosmetics composition before treatment of the hair therewith within a range from 40 to 120° C., or preferably within a range from 50 to 110° C., or preferably within a range from 60 to 100° C. The duration of the employment of elevated temperature is preferably within a range from 1 second to 3 hours, or preferably within a range from 5 seconds to 2 hours, or preferably within a range from 10 seconds to 1 hour, or preferably within a range from 20 seconds to 10 minutes.

Another subject is a cosmetic method of producing a decorative effect on hair, at least comprising the steps of:
0) if necessary dyeing or shaping the hair;
1) applying a composition comprising at least one carbodiimide of the formula ( )
   where
   n is an integer from 3 to 100, preferably from 3 to 50, or preferably from 3 to 20, or preferably from 3 to 20,
   R¹, R⁴ and R⁵ are each independently linear or branched, aliphatic or cycloaliphatic, optionally substituted groups having 1 to 100 carbon atoms, preferably 1 to 50, or preferably 1 to 20,
   to the hair.

All statements relating to the compound containing at least one carbodiimide, also called polycarbodiimide, which were made in connection with the cosmetic composition of the invention comprising the compound containing at least one carbodiimide are preferably applicable correspondingly to the method of applying this composition to hair.

As already mentioned above, the conservation of structures of the treated hair is already regarded as a decorative effect. By the method, it is preferably also possible to achieve an enhancement of the structures by the composition of the invention without further additives, such as pigments or dyes.

In addition, it is preferable that the method achieves fixing of structures in the hair. The expression “fixing of structures in the hair” shall be understood to mean the fixing of artificial structures and also of endogenous structures. Examples of artificial structures are non-endogenous or artificial hair, or other artificial elements that can be applied to the hair. Examples of endogenous structures are structures that are already present on the user's body, but have been modified in terms of their shape, position or design and are to be fixed, such as hair tresses or parts of hair tresses. Fixing shall be understood in particular to mean that the desired shape is fixed, i.e. is to be conserved over a prolonged period.

Dyeing may be any visual change to the hair known to the person skilled in the art for this purpose. Dying is preferably dying of the hair by means of appropriate hair dyes or hair tints. Any commercial products may find use for this purpose.

The shaping of the hair may be any shaping that the person skilled in the art would select for the purpose. The shaping is preferably selected from the group consisting of curling, straightening, blow-drying, combing, brushing, braiding, or a combination of at least two of these. Preferably, the shaping is straightening or curling of the hair. Curling of the hair is preferably understood to mean the implementation of a permanent wave, as typically conducted by a hairdresser. Straightening can preferably take place with commercial straightening irons and, even in the case of very frizzy hair, can lead to complete straightening of the hair.

The composition is preferably applied in an amount as typically used for hairstyling products. On application, preference is given to using an amount of cosmetic composition within a range from 1 to 10 ml, preferably within a range from 2 to 5 ml.

The application is followed, if necessary, by a contact time. The contact time is preferably a few seconds to several hours, or preferably 1 minute to 2 hours, or preferably 5 minutes to 1 hour, or preferably 10 to 30 minutes.

A film former may be used together with the cosmetic composition. The film former may either already be within the cosmetic composition or be applied as a further composition on the hair. The further composition may alternatively be applied before, simultaneously with or after the application of the cosmetic composition.

In a preferred embodiment of the method of producing a decorative effect on hair, the cosmetic composition remains at least partly thereon, especially on the applied areas of the hair. More particularly, it is preferable that the decorative effect is conserved even after repeated washing of the hair.

In a preferred embodiment of the method for producing a decorative effect on hair, in a further step 2), a further composition comprising at least one film former is applied to the hair.

Preferably, the film former is a film former as mentioned above for the hair cosmetics composition. Further preferably, the film former in step 2) is selected from the group consisting of a polyacrylate, a polyacrylamide, a polyurethane, a polyurea, a polysiloxane or a mixture of at least two of these. Preferably, the film former in step 2) is a polyurethane or a polyacrylate. Preferably, the further composition includes the film former in an amount within a range from 0.1% to 30% by weight, or preferably within a range from 0.5% to 15% by weight, or preferably within a range from 1% to 10% by weight, based on the total mass of the further composition. As already mentioned above, the further composition can be applied before, simultaneously with or after the application of the cosmetic composition. Preferably, the application of the further composition in step 2) precedes the application of the cosmetic composition in step 1).

In a preferred embodiment of the method of the invention, heating of the hair and/or the hair cosmetics composition takes place before, during and/or after step 1) or 2). The heating preferably takes place before and/or during the treatment of hair with the hair cosmetics composition of the invention in step 1) or 2). Preferred temperatures for heating the hair or the hair cosmetics composition have already been mentioned for the use of the hair cosmetics composition of the invention. Preferably, the hair and/or hair cosmetics composition to be treated before and/or during steps 1) and/or 2) is brought to a temperature within a range from 30 to 250° C., or preferably within a range from 40 to 230° C., or preferably within a range from 50 to 200° C., or preferably within a range from 60 to 180° C. The duration of the heating is preferably within a range from 1 minute to 3 hours, or preferably within a range from 5 seconds to 2 hours, or preferably within a range from 10 seconds to 1 hour, or preferably within a range from 20 seconds to 10 minutes.

In a preferred embodiment of the method of producing a decorative effect on hair further includes at least one of the following steps:
(a) heating the hair cosmetics composition before application to the hair;
(b) heating the hair before applying the hair cosmetics composition;
(c) heating the hair while applying the hair cosmetics composition;
(d) heating the hair after applying the hair cosmetics composition together with the hair cosmetics composition;
(e) combining at least two of steps (a) to (d).

The present invention will be elucidated with reference to examples, which should not be regarded as limiting. All amounts, proportions and percentages, unless stated otherwise, are based on weight and the total amount or total weight of the compositions.

### EXAMPLES

Unless stated otherwise, all analytical measurements relate to measurements at temperatures of 23° C.

The pH was determined by means of an electrode (Mettler Toledo Seven Excellence).

The combing always took place using an Ultron Carbon Line comb with a length of 22.2 cm by the side of the comb that has the broad separation of the comb teeth. With combing 3 times in succession through the hair.

The hair was run through after application of compositions using the fingers, whereby all 5 fingers were passed through the hair tress 3 times from above.

I) Test Results on Water Resistance and Wash Resistance of Hairstyles

Preparation/Shaping or Structuring of Hair Tresses (Step 0)):

For all water resistance or wash resistance tests, as summarized in table 1, European hair tresses were waved (total length 18 cm, width 8 cm, weight 1.0 g+/−0.2 g). Prior to testing, the hair tresses were washed with 0.3 g of a commercial silicone-free shampoo (Syoss “Volume Lift Shampoo”) at 38° C. for 1 minute. The hair tresses were then rinsed at 38° C. for 1 minute and combed through with a conventional comb (broad side). The hair tresses were then dried using a hairdryer at about 75° C. for 1 minute.

For each of the examples listed below and in table 1, 3 hair tresses were treated independently in the same way. The results listed in table 1 represent the average values from this triple determination.

Substances Used

The substances tested are named by the following abbreviations:
Product A: Composition that was prepared according to the instructions and the starting materials as described in example 2 from EP 2 371 873 A1 and was used in different concentrations (% by weight are given in the respective examples, each based on the total weight of the product A; the remainder to 100% by weight is made up with water in each case);
Product B: INCI: polyurethane-48, 2% by weight;
Product C: Amphomer (INCI: Octylacrylamide/Acrylates/Butylaminoethyl Methacrylate Copolymer), 100% with AMP (Amino Methylpropanol) adjusted to pH 9.6, 2% by weight.

### Step 1

### Comparative Example I-1

After preparation as described in step 0), the hair tresses were dried again by hairdryer at 75° C. for 3 minutes. Then the hair tresses were straightened 5 times at 230° C. for 3 seconds with a commercially available flat iron and then completely dried at 56% relative air humidity at 23° C. for 12 hours.

### Example I-2: Treatment with Product A

3 hair tresses were treated the same way in parallel. After the preparation of the hair tresses as described in step 0), 1 g of product A in each case, containing 2% by weight of the polycarbodiimide of the invention, based on the total amount of product A, was applied to the respective hair tress. Each of the three hair tresses were run through briefly to distribute the product A along the length of the respective hair tress. The hair tresses were combed and dried by hairdryer at 75° C. for 3 minutes. Then they were straightened with a flat iron 5 times at 230° C. for 3 seconds and then completely dried at 56% air humidity at 23° C. for 12 hours.

### Example I-3: Treatment with Product a and then with Product B or C

3 hair tresses were treated the same way in parallel. After preparation of the hair tresses as described in step 0), 0.5 g of product A was applied to each of the three hair tresses. Each of the three hair tresses was briefly run through to distribute the product A over the length of the hair tress, combed and dried by hairdryer at 75° C. for 1 minute.

Then, 0.5 g of product B or product C was applied to each of the three hair tresses to the respective hair tress. The hair tresses were briefly run through, combed and dried by hairdryer at 75° C. for 3 minutes.

Subsequently, the hair tresses were straightened 5 times at 230° C. for 3 seconds with a flat iron and then completely dried at 56% relative air humidity at 23° C. for 12 hours.

### Example I-4: Treatment with Product Product B or C and Product A

Three identical hair tresses were processed as described in example 3, except that the order of application of products A and products B or C is reversed, i.e. the respective product B or C is applied first to the hair tresses, and then product A.

### Example I-5: Treatment with a Mixture of Product a and Product B or C

Three identical hair tresses were, after preparation as described in step 0), 0.5 g of product A and 0.5 g of product B or product C are applied to the hair tress. The hair tresses are briefly run through, combed and dried by hairdryer at 75° C. for 3 minutes. Then they are straightened with a flat iron 5 times at 230° C. for 3 seconds and then completely dried at 56% air humidity at room temperature for several hours.

Water Resistance Test

Each of the hair tresses treated in examples I-1 to I-5 was individually dipped completely into a 2 liter water bath heated to 38° C. for 30 seconds, left to drip for 10 seconds and left to dry completely at room temperature in air for 24 h. The width of the tress was then measured with a ruler (1 millimeter scale).

Wash Resistance Test:

The hair tresses from step 1) were washed with 0.3 g of the commercially available silicone-free shampoo (Syoss Volume Lift Shampoo) at 38° C. for 1 minute. The hair tresses were then rinsed for 1 minute in a water bath heated to 38° C. for 30 seconds and combed through with a comb (broad side). Subsequently, the hair tresses were dried at room temperature for 24 h. The width of the tress was then measured with a ruler (1 millimeter scale).

Results:

The results are summarized in table 1.

Example I-1: Treatment without product (comparative example)

Example I-2-1: Treatment with product A (according to the invention)

Example I-3-1: Treatment with product A and product B (according to the invention)

Example I-3-2: Treatment with product A and product C (according to the invention)

Example I-4-1: Treatment with product B and product A (according to the invention)

Example I-4-2: Treatment with product C and product A (according to the invention)

Example I-5-1: Treatment with a mixture of product A and product B (according to the invention)

Example I-5-2: Treatment with a mixture of product A and product C (according to the invention)

TABLE 1
compositions tested

| | | Width | | | |
|---|---|---|---|---|---|
| | | before/after | Width after | Width after wash resistance test | |
| | | straightening | water resistance | [cm] | |
| Example | Products used | [cm] | test [cm] | Washed once | Washed twice |
| I-1 | — | 8/1.4 | 5 | >9 | >9 |
| I-2-1 | A | 8/1.4 | 1.7 | 3.5 | 4.4 |
| I-3-1 | A then B | 8/1.4 | 2.1 | 1.7 | 1.9 |
| I-3-2 | A then C | 8/1.4 | 2.3 | 8.7 | — |
| I-4-1 | B then A | 8/1.4 | 1.4 | 1.7 | 2.4 |
| I-4-2 | C then A | 8/1.4 | 4.1 | 7.3 | — |
| I-5-1 | Mixture A + B | 8/1.4 | 1.3 | 2.0 | 2.4 |
| I-5-2 | Mixture A + C | 8/1.4 | 3.1 | 7.3 | 7.5 |

As can be seen in table 1, the treatment of hair tresses by product A (example I-2-1), which is a polycarbodiimide composition of the invention, has a structure-conserving effect on the treated hair tresses, since the width of the hair led to lower broadening of the hair tresses even after treatment in water and the treatment with shampoo than without the application of carbodiimides or using other noninventive compositions.

Only a broadening of 1.21 times that of the treated, i.e. straightened, hair tresses after water treatment is recorded in example I-2-1. The subsequent washing of the hair tresses with shampoo led to broadening by 2.5 times of the original hair tress in the first washing step and by 3.14 times in the second washing step. The best results in terms of conservation of structure of the straightened hair were achieved with the aid of a combination of product A with product B. The best results were achieved both in the combination of the two products (example I-5-1) in one step and in the successive application of product A and then product B (example I-3-1), compared to combinations with product C and compared to sole use with product A, as can be seen in table 1.

In contrast, the untreated hair from comparative experiment I-1, which was not treated with any of the products A, B or C, underwent broadening by 3.5 times and hence became broader than all other examples after the wash. Smoothing of the hair with a flat iron thus leads, without structure-conserving measures, even after one wash, virtually back to the originally curly hair, while the treatment with a composition of the invention leads to a structure-conserving effect, at least on contact with water. Washing with the shampoo resulted in broadening of the hair to a value greater than the baseline before straightening.

II) Test Results on Moisture Resistance of Hairstyles

Preparation/Shaping or Structuring of Hair Tresses (Step 0)):

For all moisture resistance tests, as summarized in table 2, Kerling hair tresses identified as “highly bleached, Color 10/0” with total length 21 cm, free hair 19 cm, width of the tress 1.5 cm, weight 1.0 g+/−0.2 g, were used. Prior to testing, the hair tresses were washed with 0.3 g of a commercial silicone-free shampoo (Syoss “Volume Lift Shampoo”) at 38° C. for 1 minute. The hair tresses were then rinsed at 38° C. for 1 minute and combed through with a conventional comb (broad side).

For each of the examples listed below and in table 2, 2 hair tresses were treated independently in the same way. The results listed in table 2 represent the average values of this double determination.

### Step 1

### Comparative Example II-A1

After preparation as described in step 0) under II), the hair tresses were dried with the aid of a hairdryer at 75° C. for 3 minutes. Then they were straightened with a flat iron 5 times at 230° C. for 3 seconds and then completely dried at 56% relative air humidity at 23° C. for 12 hours. This treatment according to step 0) and step 1) of shampooing, rinsing, combing and drying was repeated 10 times for each hair tress.

### Example II-A2: Treatment with Carbodiimide

After preparation of the hair tresses as described in step 0) under II), 1 g of product A (5% by weight in water) was applied to the wet hair tress. The hair tress was briefly run through to distribute the product as evenly as possible over the length of the hair. The hair tress was combed and dried by hairdryer at 75° C. for 3 minutes. Thereafter, the hair tresses were straightened 5 times at 230° C. for 3 seconds with a flat iron and then completely dried at 56% air humidity at 23° C. for 12 hours. This treatment according to step 0) and step 1) of shampooing, rinsing, combing and drying was repeated 10 times for each hair tress.

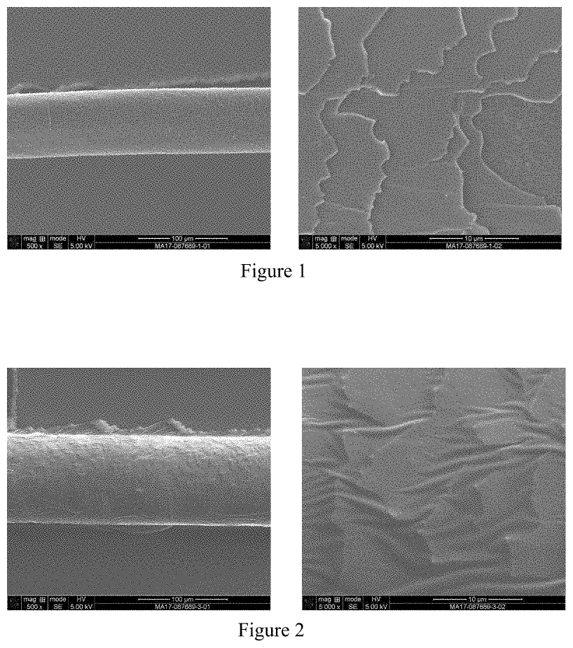

One of the hair tresses from each of comparative example II-A1 and example II-A2 was examined with the aid of an FEI Magellan400 scanning electron microscope (SEM) (to DIN ENISO/IEC 17025 method). One hair from a hair tress according to comparative example II-A1 is shown in FIG. 1 and one from example II-A2 in FIG. 2. The images from FIGS. 1 and 2 were created in secondary electron contrast at an acceleration voltage of 5 kV with two magnifications of 500× and of 5000×.

It is clearly apparent from FIG. 1 that the product A has formed a film around the hair. In particular, this film covers hydrophilic areas (visible on the SEM image as thickening of the layer) of very stressed and damaged hair. The scaly structure of the untreated hair is evenly covered by the film as a smoother structure. For that reason, the hair tresses that have been treated with the composition of the invention in the form of product A did indeed show significantly more pleasant tactile properties and distinctly increased combability. The values for the tactile properties are shown in table 2. A scale was used here from 1 (very rough) to 2 (rough), 3 (smooth) to 4 (very smooth). Compared to the very rough hair (1) without treatment with the product A of the invention, the hair treated with product A of the invention felt very smooth (4).

To demonstrate this effect, the coefficients of friction of the bleached hair from comparative experiment II-A1 and example II-A2 were performed.

Preparation of Hair Tresses to Determine the Coefficient of Friction (Step 0)

Before the tests, the hair tresses were pretreated as described in step 0) under II).

### Comparative Example B1

Subsequently, the 2 hair tresses were completely dried at 56% air humidity at 23° C. for at least 12 hours.

### Example B2 and B3

After the preparation of the hair tresses as described in step 0), 1 g in each case of product A was applied to the wet hair tress as an aqueous solution (2% by weight for example B2 or 1% by weight for example B3). The hair tresses were each briefly run through to distribute the product over the length of the hair tresses. The respective hair tress was combed and completely dried at 56% air humidity at 23° C. for at least 12 hours.

For each of the examples listed below and in table 2, 4 hair tresses were treated independently in the same way.

Results

The coefficient of friction was measured using an MTT175 Tensile Tester from Dia-Stron. For each hair tress, the coefficient of friction was measured in 2 directions: once from the hair root to the hair tip (COF Out) and once from the hair tip to the hair root (COF Return). The values were measured on 4 hair tresses in each case. The mean and standard deviations are summarized in table 2.

TABLE 2
Coefficient of friction and tactile values
of hair treated with and without product A

| | COF Out | | COF return | | |
|---|---|---|---|---|---|
| | | Standard | | Standard | Tactile |
| Example | Mean | deviation | Mean | deviation | properties |
| B1 | 1.082E−02 | 4.972E−04 | — | — | 1 |
| B2 with 1% | 9.283E−03 | 2.498E−04 | 9.984E−03 | 8.220E−04 | 4 |
| by weight of | | | | | |
| product A | | | | | |
| B3 with 2% | 9.254E−03 | 6.831E−04 | 1.046E−02 | 1.297E−03 | 4 |
| by weight of | | | | | |
| product A | | | | | |

It was shown that the coefficient of friction of bleached hair was significantly reduced by treatment with carbodiimides.

III) Test Results with Brazilian Hair

### Example III-C: Treatment of Brazilian Hair with Inventive Composition in the Form of Product A

Step 0)

Prior to the tests, the hair tresses, in this case Kerling “Brazilian Natural Hair”, with total length 20 cm, free hair 18 cm, were washed with 0.3 g of a commercial silicone-free shampoo (Syoss “Volume Lift Shampoo”) at 38° C. for 1 minute. The hair tresses were then rinsed at 38° C. for 1 minute and combed through with a wide comb.

For each of the examples listed below and in table 3, 2 hair tresses were treated independently in the same way. The values in table 3 are the average values of 2 hair tresses each.

### Comparative Example C1

Subsequently, the hair tresses were treated as follows:
C1-1: air-dried: the hair tresses are completely dried at 56% air humidity at 23° C. for at least 12 hours
C1-2: blow-dried: dried with the aid of a hairdryer at about 75° C. for 3 minutes, then completely dried at 56% air humidity at 23° C. for at least 12 hours
C1-3: blow-dried and straightened: dried by hairdryer for 3 minutes at 75° C., then they were straightened with a flat iron 5 times at 230° C. for 3 seconds and then completely dried at 56% air humidity at 23° C. for 12 hours

### Example C2 (C2-1 to C2-5)

After preparation of the hair tresses as described in step 0), 1 g in each case of product A as a solution having different polycarbodiimide contents of 5% by weight, 2% by weight or 1% by weight active in water was applied to the respective hair tresses. The hair tresses were briefly run through to distribute the product over the length of the hair tress. The hair tress was combed and then air-dried, blown, or blown and straightened as described above, resulting in the following examples:
C2-1: 5% by weight of product A in water, then air-dried: the hair tresses are completely dried at 56% air humidity at 23° C. for at least 12 hours
C2-2: 2% by weight of product A in water, then air-dried: the hair tresses are completely dried at 56% air humidity at 23° C. for at least 12 hours
C2-3: 1% by weight of product A in water, then air-dried: the hair tresses are completely dried at 56% air humidity at 23° C. for at least 12 hours
C2-4: 5% by weight product A in water, then blow-dried: dried with the aid of a hairdryer for 3 minutes at about 75° C., then completely dried at 56% air humidity at 23° C. for at least 12 hours
C2-5: 5% by weight of product A in water, then blow-dried and straightened: dried by hairdryer for 3 minutes at 75° C., then they were straightened with a flatiron 5 times at 230° C. for 3 seconds and then completely dried at 56% air humidity at 23° C. for 12 hours.

Results

The width of the tresses from comparative examples C1-1 to C1-3 and examples C2-1 to C2-5 were determined with a ruler (1 millimeter scale). Thereafter, the tresses were stored for 24 hours in an HCP 246 climate-controlled cabinet from Memmert GmbH at 29° C. and 90%, and their length was measured again.

TABLE 3
Results for the treatment of Brazilian hair with compositions of the
invention of different concentrations compared to untreated hair

| | | | Width after |
|---|---|---|---|
| | | Starting | 24 hours at |
| | | width | 90% air humidity |
| Example | Treatment | [cm] | [cm] |
| C1-1 | Without product A, air-dried | 7.0 | 8.0 |
| C2-1 | With product A | 3.0 | 3.75 |
| | (5% by weight), | | |
| | air-dried | | |
| C2-2 | With product A | 4.0 | 4.25 |
| | (2% by weight), | | |
| | air-dried | | |
| C2-3 | With product A | 5.3 | 6.4 |
| | (1% by weight), | | |
| | air-dried | | |
| C1-2 | Without product A, blow-dried | 8.0 | 8.0 |
| C2-4 | With product A | 2.7 | 2.7 |
| | (5% by weight), | | |
| | blow-dried | | |
| C1-3 | Without product A, | 4.0 | 8.0 |
| | blow-dried | | |
| | and straightened | | |
| C2-5 | With product A | 2.0 | 3.7 |
| | (5% by weight), | | |
| | blow-dried and straightened | | |

The hair tresses treated with the composition of the invention in the form of product A (examples C2-1, C2-2, C2-3, C2-4, C2-5) have a much lower volume in a humid atmosphere than the hair tresses not treated with a composition of the invention (C1-1, C1-2, C1-3). These results are consistent with the results on water resistance (from examples I-1 to I-5-2). It was thus surprisingly shown that the treatment of hair with a composition of the invention results in hydrophobing of the hair surface. In particular, it has been shown that the composition of the invention forms a closed film on the hair, as in the comparison of FIG. 1, which shows a hair without treatment with a composition of the invention, and FIG. 2, which shows a hair treated with a composition of the invention, containing 5% by weight of product A. The film formed and shown in FIG. 2 is able to protect the hair from external influences such as humidity, water, shampoo, heat, etc., and so a hairstyle created is retained for longer even though it has been exposed to these influences that otherwise destroy hairstyles, as has been shown by the examples.

The present invention is elucidated in detail with reference to the following figures, but without being limited thereto. The figures show:

FIG. 1: Two illustrations (500- and 5000-fold magnification) of a hair without treatment with a composition of the invention in a scanning electron microscope

FIG. 2: Two illustrations (500- and 5000-fold magnification) of a hair after treatment with a composition of the invention in a scanning electron microscope

FIG. 1 shows a hair from a hair tress as treated according to example C1-1 in the form of a scanning electron micrograph in secondary electron contrast at an acceleration voltage of 5 kV produced. The FEI Magellan400 scanning electron microscope was used in two different magnification settings. The illustration in FIG. 1 on the left-hand side has been produced with 500-fold magnification and the illustration on the right-hand side of FIG. 1 has been produced with 5000-fold magnification. In both images, scaly structures are clearly apparent, which have quite sharp edges in their transitions from one scale to the next.

FIG. 2 shows a hair from a hair tress as treated according to example C2-1 in the form of a scanning electron micrograph in secondary electron contrast at an acceleration voltage of 5 kV. The FEI Magellan400 scanning electron microscope was used in two different magnification settings. The illustration in FIG. 2 on the left-hand side has been produced with 500-fold magnification and the illustration on the right-hand side of FIG. 2 has been produced with 5000-fold magnification. It is clearly apparent that the scaly structure of the hair, as shown in FIG. 1, is surrounded by a closed film of the composition of the invention in the form of product A. The film has the effect here that it significantly attenuates and softens the sharp edges of the individual scales, as can be seen in the right-hand illustration in FIG. 2. The film lies like a cloth over the hair structure and even forms wrinkles there, as is clearly apparent in the right-hand illustration of FIG. 2.

1. A hair cosmetics composition comprising at least one compound containing at least three carbodiimide groups and having a structure conforming to the formula (I)
R⁴—[R¹—N═C═N]ₙ—R⁵ (I)
where
n is an integer from 3 to 100,
R¹, R⁴ and R⁵ are each independently linear or branched, aliphatic or cycloaliphatic, optionally substituted groups having 1 to 100 carbon atoms.

2. The hair cosmetics composition as claimed in claim 1, wherein the compound containing at least three carbodiimide groups has the general formula (II): 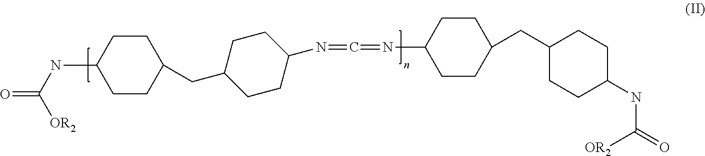
in which R₂ and R₃ are independently selected from a radical derived from a monoalkoxy poly(ethylene glycol) of the general formula (III)
R⁶—O—(CH₂—CH₂—O)ₘ—H (III)
with R⁶=C₁ to C₃₀ alkyl or acyl group and m=4 to 30,
a radical derived from C₁ to C₃₀ alcohol, and a radical derived from C₁ to C₃₀ monoalkoxy ethylene glycol.

3. The hair cosmetics composition as claimed in claim 2, wherein the composition comprises the compound containing at least three carbodiimide groups of the formula (I) or (II) within a range from 0.01% to 50% by weight.

4. The hair cosmetics composition as claimed in claim 1, wherein the composition further comprises at least one of the following components:
1) at least one cosmetically active substance, one or more cosmetic auxiliary substances, or both;
2) at least one solvent or thinner; and
3) at least one film former.

5. The hair cosmetics composition as claimed in claim 4, wherein the at least one film formers comprises a nonionic polymer, an anionic polymer, an amphoteric polymer, a cationic polymer, or a mixture of at least two of these.

6. The hair cosmetics composition as claimed in claim 4, wherein the at least one film former comprises a polyurethane obtainable by reacting one or more water-insoluble, non-water-dispersible, isocyanate-functional polyurethane prepolymers A) with one or more amino-functional compounds B).

7. The hair cosmetics composition as claimed in claim 4, wherein the at least one compound containing at least three carbodiimide groups and the at least one film former are in a weight ratio of 2:1 to 1:4.

8. The hair cosmetics composition as claimed in claim 1, wherein the composition is in a form selected from the group consisting of a pump spray, an aerosol, a gel, a foam, a mousse, a lotion, a wax, a pomade, an oil, a milk, an emulsion, an aqueous solution, and a cream.

9. The hair cosmetics composition as claimed in claim 1, wherein the cosmetic composition is formulated for maintenance of artificially shaped structures of the hair.

10. A cosmetic method of producing a decorative effect on hair, comprising:
applying a composition to the hair, where the composition comprise at least one carbodiimide of the formula (I)
R⁴—[R¹—N═C═N]ₙ—R⁵ (I)
where
n is an integer from 3 to 100,
R¹, R⁴ and R⁵ are each independently linear or branched, aliphatic or cycloaliphatic, optionally substituted groups having 1 to 100 carbon atoms.

11. The method as claimed in claim 10, further comprising
dyeing or shaping the hair prior to applying the composition to the hair.

12. The method as claimed in claim 10, wherein the composition remains at least partly on the hair after application thereto.

13. The method as claimed in claim 10, wherein, in a further step 2), a further composition comprising at least one film former is applied to the hair.

14. The method as claimed in claim 13, wherein step 2) precedes step 1).

15. The method as claimed in claim 13, wherein the hair is heated before steps 1) or 2, during steps 1) or 2), after steps 1) or 2), or a combination thereof.

16. A hair cosmetics composition comprising:
0.5 wt % to 10 wt % based on a total weight of the composition of a compound containing at least three carbodiimide groups and having a structure conforming to the formula (I)
R⁴—[R¹—N═C═N]ₙ—R⁵ (I)
where
n is an integer from 3 to 10,
R¹ is a linear or branched, aliphatic or cycloaliphatic, group having 4 to 20 carbon atoms, and
R⁴ and R⁵ independently comprise a radical comprising at least one urethane group and at least one group that is a radical derived from a C₁ to C₃₀ alcohol or a C₁ to C₃₀ monoalkoxy ethylene glycol;
0.01 wt % to 10 wt % based on a total weight of the composition of a film former comprising a polyacrylate, a polyacrylamide, a polyurethane, a polyurea, a polysiloxane, or a mixture of at least two of these; and
water.
